(19) United States
(12) Patent Application Publication
(10) Pub. No.: US 2022/0080071 A1
(43) Pub. Date: Mar. 17, 2022

(54) Apparatus, System, and Method for Enabling Robots to Autonomously Disinfect In-door Spaces Using Remote-controlled Automated Shades

(71) Applicants: Christian Wilhelm Fritz, Menlo Park, CA (US); Robert Steven Bauer, Portola Valley, CA (US)

(72) Inventors: Christian Wilhelm Fritz, Menlo Park, CA (US); Robert Steven Bauer, Portola Valley, CA (US)

(21) Appl. No.: 17/446,561

(22) Filed: Aug. 31, 2021

Related U.S. Application Data
(60) Provisional application No. 62/706,850, filed on Sep. 14, 2020.

(51) Int. Cl. A61L 2/24; A61L 2/10; A61L 2/22; E06B 9/68

(52) U.S. Cl. CPC A61L 2/24; A61L 2/10; A61L 2/22; E06B 9/68; E06B 2009/6845; A61L 2202/11; A61L 2202/14; A61L 2202/15; A61L 2202/16; A61L 2202/25

(57) ABSTRACT

We present an invention that enables autonomous disinfection robots to perform their duties significantly more autonomously than previously possible. Because disinfection mechanisms, such as UV-C light and chemical disinfectant mists, are harmful to humans, robots that provide such disinfection functionality in interior spaces require people to be kept out of the areas of disinfection. Human help is currently required for opening and closing doors for the robot to secure the space during disinfection. We describe a system and method for enabling robots to perform this task safely without human help. In one embodiment this is achieved by mounting a radio-controlled, motorized shade on the door-frame of the room to be disinfected, and enabling the robot to autonomously roll that shade up and down. The shade stops significant amounts of the dangerous UV-C light or chemical disinfectants from leaving the room, enabling a robot to disinfect multiple rooms without human help.

### TECHNICAL FIELD

The present disclosure is generally related to mobile robot technology, including techniques for autonomous disinfecting indoor spaces where the system includes shades controlled by the robot to open and close said spaces.

### BACKGROUND

The spectrum of ultraviolet light is broken down into three sections: A, B, and C. While UV-A and UV-B light occurs naturally on earth, UV-C light is filtered by our atmosphere and is hence not naturally found on earth. UV-C light is furthermore of higher energy than UV-A and UV-B. Because pathogens on earth do not naturally get exposed to UV-C light and because of its high energy, UV-C light is germicidal, a property it is known for over a hundred years. For these germicidal properties, UV-C light has been used in hospitals for over sixty years to disinfect various spaces, including operating rooms and patients rooms, especially after discharge. To do this, a cart full of UV-C lamps typically is wheeled into the room to be disinfected, plugged into the wall, and left there for a certain amount of time (typically in the range of 5 to 45 minutes).

However, it is important to notice that UV-C light is dangerous to humans, damaging the skin and retina and causing cancer. The Occupational Safety and Health Administration therefore recommends that humans only get minimally exposed to this light. Hence, while these lamps are turned on, humans must vacate the room to remain safe. At the same time it is necessary for the UV-C light source to be moved around the room in order to avoid shadowing, where pathogens remain safe from the light. This is especially true for en-suite bathrooms, as well as larger rooms with multiple beds and curtains.

To address this challenge, multiple people have conceived of the idea to motorize the cart of UV-C lamps, i.e., creating an electromechanically actuated, mobile robot that can move around autonomously or via teleoperation inside the space to be disinfected. Using either a map or purely based on sensors, such autonomous UV-robots can self-navigate the room and carry the light around to shine on all exposed surfaces. These robots are furthermore able to navigate the facilities these rooms are located in, e.g., the halls of a hospital, to autonomously navigate to and from the rooms and other spaces they are tasked with disinfecting.

The Problem

While these robots address a significant problem with manual UV-carts, they typically still require human assistance, namely to open and close doors for them. This is necessary when temporarily vacating the room is possible but vacating the space in front of the room, for the required amount of time, is not, e.g., a corridor or hallway. This is the case in many current applications for these disinfection robots, including but not limited to hospitals, hotels, nursing homes, and schools. Hence, in its current implementation, the robot is hailed to a room, where it is met by a person. The person then opens the door for the robot, the robot moves inside, the person closes the door behind the robot and only then the robot can start disinfecting the room. Once the robot is done, the person opens the door again and the robot comes out.

This reliance of the robot on a human significantly reduces its ability to be of service and help in disinfecting a lot of spaces in a hospital or other environments. These robots are capable of safely navigating from room-to-room in such an indoor environment and as such could autonomously disinfect multiple rooms in a row or at various times of the day or night if only they could do so without letting UV-C light come out of the room or space they are disinfecting.

Unfortunately it is exceedingly difficult for autonomous mobile robots to open and close doors by themselves in the way humans do: pressing down on a door handle or grabbing and turning a door knob, when pulling/pushing while simultaneously moving backwards/forwards with the motion of the door. Hence, the seemingly obvious solution of having the robot do just that is not technically feasible or yet, at least not reliably.

### BRIEF DESCRIPTION OF THE SEVERAL VIEWS OF THE DRAWING

FIG. 1a shows an embodiment where the motorized shade is mounted inside the door frame. In the picture on the left, the share is rolled up, i.e., in the “open” position. In the picture on the right, the shade is rolled down, i.e., in the “closed” position. The shade covers the entire doorway when in the closed position.

FIG. 1b shows a similar embodiment as FIG. 1a but with the motorized shade mounted outside the door frame. In the picture on the left, the share is rolled up, i.e., in the “open” position. In the picture on the right, the shade is rolled down, i.e., in the “closed” position. The shade covers the entire doorway when in the closed position.

FIG. 1c shows a similar embodiment as FIG. 1a but with the motorized shade mounted above the door frame. In the picture on the left, the share is rolled up, i.e., in the “open” position. In the picture on the right, the shade is rolled down, i.e., in the “closed” position. The benefit of this embodiment over the ones shown in FIGS. 1a and 1 b is that the shade itself can be wider than the door frame itself, making it even easier to prevent the disinfectant, e.g., UV-C light, to escape the indoor space to be disinfected by the robot, in this case a bathroom, even when the door to that room is open.

FIG. 2 shows a workflow diagram of an embodiment including a motorized shade, e.g., the one shown in FIG. 1c. The figure shows the steps in order, starting at the top, with arrows showing the direction in which the steps of the workflow proceed.

### SUMMARY OF INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefits and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

In the following description, for purposes of explanation, numerous specific details are set forth in order to provide a thorough understanding of the present invention. It will be evident, however, to one skilled in the art that the present invention may be practiced without these specific details. The present disclosure is to be considered as an exemplification of the invention, and is not intended to limit the invention to the specific embodiments illustrated by the figures or description below.

The System

We propose to solve this problem by using automated, communicating shade or shades that are in the doorways or hallways leading into a room or space or area to be disinfected, and enabling the robot to remotely and autonomously control these shades. The automated shades can be mounted on or inside all door-frames or placed by the robot onto a mounting mechanism such as a rail or hook. This effectively gives the robot the capability it needs, namely to safely disinfect a room or space or area without any human assistance. For disinfection of a room, the idea is that the robot can travel to the room where the door was left open, enter, and then close the shade or shades that had been mounted or placed in the doorway and thereby block any significant amount of UV-C light or other disinfectant, such as chemical or electrostatic or other chemical spray/fog, from leaving the room or space being disinfected. Once the shade(s) are closed, the robot can disinfect the room without harm to any potential passers-by outside the room or space. When it is finished, it can open the shades again, leave the room, and go on to its next task. As indicated, this system can be used for disinfecting both rooms and spaces larger than a single room where the automated, communicating shade or shades are mounted or placed in a hallway to provide blocking of harmful UV-C light or other disinfectant from human exposure. In another embodiment, the shades can be arranged in any space, including an open-space like a warehouse, such that when lowered or closed, define discrete, closed-off areas in which the robot can perform disinfection.

### The Automated Shades

At a minimum, the automated shades comprise a shade, a motor, a control device, and a communication device. In many embodiments the automated shades will further involve other mechanical components to connect motor and shade, mounting the motor, control device and communication device in a case, and/or on the wall or doorframe. The shade is directly or indirectly actuated or moved by the motor. The motor is controlled by the control device. The control device, which may be, but not limited to, a transceiver that receives control signals broadcast by the robot or by a network such as the Internet to which the robot may be wireless connected. The control signal broadcast by the robot contains encoded control commands that actuate the motor to which the shade is connected. The transceiver may generate a message packet containing the state of the control device which is transmitted to the robot or to the network infrastructure to which the robot is wirelessly connected. In this way the disinfection system, comprised of the disinfection robot and automated shade, can ensure the position of the shade as being (a) in place for human protection during robot disinfection, (b) in motion, or (c) retracted so the robot can exit the room or space that has been disinfected.

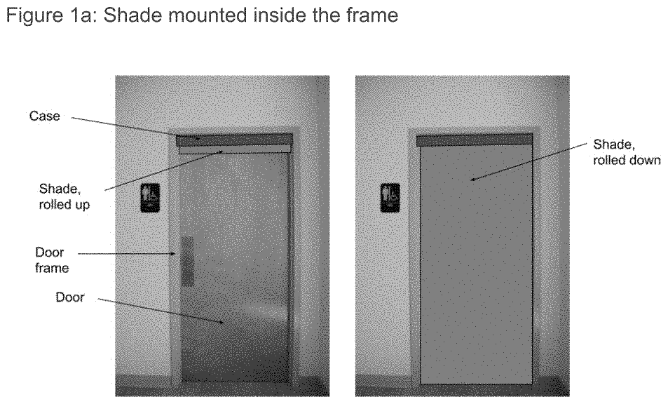

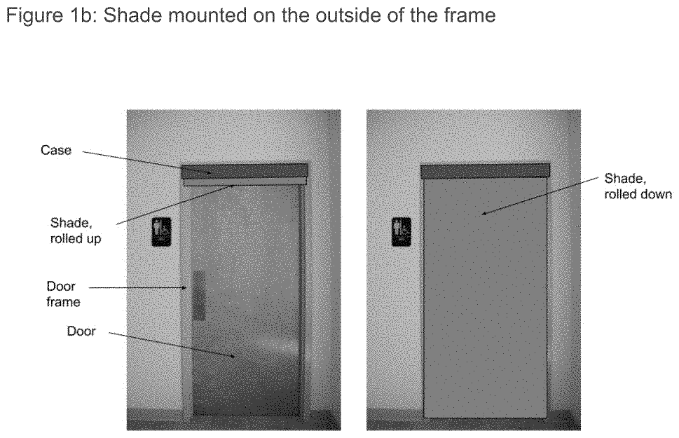

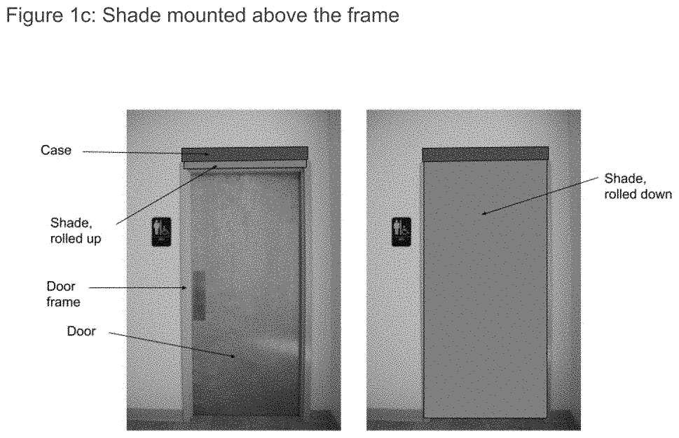

In one possible embodiment the shades are so-called roller shades, i.e., made of a flexible material such as cloth or thin plastic, that is rolled up and down using a roll propelled by a motor. FIGS. 1a-1c show some of the possible mounting options for one embodiment of such roller shades and their case. In all these examples, the shades need to be mounted on the side of the door in which the door doesn't swing open, as to allow the door to be open while lowering the shades. On sliding doors the shades can be mounted on either side.

In another possible embodiment, the shades are rigid planes, like a door, that swing open or closed just like the door itself. In another embodiment the existing door in the door frame itself is motorized and controlled by the robot.

When the robot is responsible for disinfecting a multitude of rooms or spaces, then such shades can be mounted on the door frames leading into each of those rooms or spaces. In that case, the robot is equipped with a mechanism to individually control these shades. In one possible embodiment the robot uses the same radio control mechanism to control the shades but sends different signals to the ‘addresses’ of each individual shade's receiver or communicates at different frequencies where the transceivers connected to the shades operate at individually unique frequencies.

The case of the shades are mounted in such a way that the actual shade, when rolled down to close the opening, is very close to the door frame, ideally touching it or within a few millimeters, as to prevent any UV-C light from coming out through the crack between the shades and the door frame.

In one embodiment, the shades have signage on the outside indicating that disinfection is in progress and that a person is not to enter. In case of rolled shades it would be awkward for a human to try and push them aside to enter the room. This helps remind a person that this is not a good idea. This is a benefit of using shades that are distinct from the regular door, since the shades themselves serve as indication to persons that they are not supposed to enter when they are closed. This is not the case when the door itself is used to prevent UV-C light or any other used disinfectant from getting into the area outside the room or space, since people may not be able to distinguish easily whether the door is closed for disinfection purposes or another reason.

In one embodiment, the shades are made of a transparent plastic that filters UV-C light but lets other parts of the light spectrum through, thereby enabling people to notice and see the robot performing the disinfection from outside the room or space. This can serve as an additional reminder not to enter. Since UV-C light does not easily penetrate most materials, not even glass, there are a multitude of possible materials that could be utilized for such an embodiment.

In another possible embodiment, the shades are curtains that are drawn sideways by a pulley mechanism, actuated by a motor.

In some embodiments the shades or their case will further be equipped with a motion sensor that can detect if someone or something moves or perturbed the shade. When this happens, the shade's control device can send a signal to the robot or the Internet to which the robot may be wirelessly connected to let the robot know that a person may be trying to enter. The robot can then turn off the UV-C light as an additional safety precaution to protect the entering human from the harmful light.

### The Robot

Robots that perform disinfection using UV-C light or other disinfectants, such as chemical or electrostatic chemical spray/fog, have been proposed by several inventors before. The invention presented here applies to the usage of any such robot and can be of a variety of sizes, from low-profile robots that focus on disinfecting the floor to robots that are taller than an adult, the robot carrying UV-C lamps vertically to shine in all directions or only some, for example, or carrying chemicals for spraying or fogging with a mist, whether electrostatic or not. In a special case of this invention, the “robot” is stationary, mounted in the room and consists merely of a UV-C lamp with a control device which may also contain sensors that can determine when it is safe to disinfect, i.e., when no humans are present and the shades can be lowered to keep UV-C light away from the area outside the room or space. In another special case of this invention, the “robot” is stationary, mounted in the room, and consists merely of chemicals or an electrostatic or other sprayer/fogger.

In the canonical embodiment of this invention, the robot is an autonomous mobile robot on wheels and carrying a computer and sensors that allows it to autonomously navigate the facility it is operating in.

In the case where the automated shade or shades are not permanently mounted on or inside the door-frame or hallway, the robot would have the capability to carry its shade and place it on a mounting mechanism such as a rail or hook in the doorway or hallway area when it enters the room or hall. Once properly positioned, the robot would use an actuator to raise the shade to the top of the door-frame or ceiling area, then move backwards to be over the mounting mechanism, and then retract the actuator to lower the shade in place on the mounting mechanism in the room's doorway or hall. Because the retracted shade and its possible housing dimension would be at least the width of a doorway, the robot would contain an electromechanical mechanism to rotate the shade from its ‘carry’/transport position to an orientation horizontal with the door jam or hallway ceiling; for example, if the shade and its possible housing are transported in a vertically oriented position perpendicular to the mobile robot base, the mechanism would rotate the shade and its possible housing 90 degrees around the forward-backward axis of the robot. While this increases the complexity and cost of the robot, it reduces the facility implementation cost for fully automated disinfection by the ratio of number of robots needed for disinfection to the number of rooms and spaces being disinfected.

### The Method

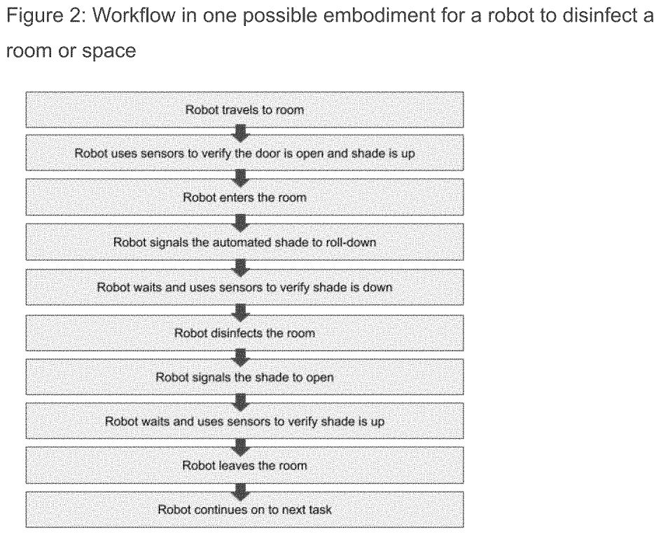

There are several possible embodiments for the methods used by the robot to remotely control the automated shades. FIG. 2 shows one possible method of operation for such a robot disinfection system comprised of the robot and the automated shades in this invention. In this embodiment, the motor driving the shades is connected to a radio controlled device, such as those using Bluetooth, Wifi, cellular data or similar connections. Accordingly the robot is equipped with a corresponding communication device allowing it to directly signal said receiver on the shades. In another embodiment, the robot communicates with said received indirectly via a communication network, either one local to the facility it is operating in or via the Internet.

There are several possible embodiments for the methods used by the system to ensure that the control of the automated shades did in fact place the shade or shades in the proper position and orientation to effectively block the vast majority or all of the harmful disinfectant, be it UV-C or chemical. In one such embodiment the robot uses a light-based sensor, e.g., lidars or cameras, to detect whether the shade is up or down and/or whether the door is opened or closed. In another possible embodiment the robot uses sound-based, for instance ultrasound, sensors to detect this. In another embodiment the transceiver in the automated, communicating shade generates a message packet containing the state of the shade (e.g., open/up or closed/down). This signal is transmitted to the robot either directly or via a network infrastructure to which the robot is wirelessly connected, e.g., the Internet. In this way the disinfection system, comprised of the disinfection robot and automated shade, can ensure the position of the shade as being (a) in place for human protection during robot disinfection, (b) in motion, or (c) retracted so the robot can exit the room or space that has been disinfected. One possible method for the data in the message packet to ensure this result is to transmit the number of rotations of the motor to which the shade is connected; this can be a the count of the control signals sent to the motor or a count of an encoder placed on or about the shaft being rotated by the motor, the shade being attached to the shaft. This data could also come from a device, such as but not limited to an optical sensor or mechanical ‘feeler’ attached to a potentiometer, that monitors the total diameter of the shade plus shaft, where the bare shaft value indicates that the automated shade is fully deployed and where the shade thickness plus shaft diameter value indicates that the automated shade is fully retracted.

There are various methods and embodiments to ensure that the room is not occupied by humans when the shade is deployed. For example, the robot can contain sensors, like infrared or motion detectors, that can measure the presence of a human or human activity. In other situations, sensors may not be necessary on the robot, be it mobile or ‘stationary’ (both cases referred to as ‘robot’) or may not be present in the room or space at all. For examples: (1) the sensors could be separate from the ‘robot’ or the automated shade control device. An example of this case is when every room in a facility has a web-connected camera or motion sensor that is used to remotely monitor persons and/or movements in the room (e.g., Alzheimer patient monitoring.) (2) There may be no sensors at all and the disinfecting ‘robot’ is remotely controlled when the room or space should be unoccupied; for examples for a common area of a nursing home in the evening, or a patient or operating room in a hospital when turnover is scheduled. In this case, if a person is mistakenly in the room, they can (a) exit, (b) override the control signal that is being sent from the remote teleoperation sub-system, or (c) alert someone of the situation (this assumes a delay between shade deployment and teleoperator initiation of disinfection).

When the robot is responsible for disinfecting a multitude of rooms or spaces, the robot or robots will be scheduled and monitored by a sub-system that also provides a status report on each robot's location and state (e.g., moving, controlling a shade, disinfecting). Rather than just a general robot fleet control scheme, this sub-system is specifically designed for integrated control and optimization of the operation of the automated shades for safety, efficiency, and disinfection effectiveness. We now describe one possible method of operation for a disinfection system of a robot or multiple robots disinfecting a multitude of rooms or spaces with automated shades for each of these multiplicity of rooms or spaces. In this embodiment, the integrated process involves the method for individual room or space disinfection, as was illustrated in one embodiment in FIG. 2, within a process that embodies the priorities for disinfection of each room or space. This process may be controlled by a disinfection task queue and accounts for the availability of the ensemble of disinfection robots. The priority of tasks undertaken by the robots in the queue can vary from a simple temporal ordering of requested disinfection tasks (i.e., the first request for a room or space to be disinfected is executed before the immediate next disinfection request that follows it, and so forth) to a complex arrangement of externally provided constraints or schedules. For examples, in a hospital, (1) all scheduled operating rooms (OR) disinfection tasks will be completed before any other room disinfection task that is requested during the execution of OR disinfection tasks, or (2) emergency department disinfection tasks will all be completed before any patient room disinfection except when a OBGYN patient is in active labor). The multitude of available robots will be monitored and sent commands for each task based on this task queue, with automated monitoring not only of each robot's disinfection completion status, but crucially, the status of the automated shade associated with the room or space in which the robot disinfection was completed. Only after receipt by the task queue sub-system of the signal from the automated shade that it had been fully retracted will the sub-system send a new task command to the robot to proceed to another room or space for disinfection, that location being determined by the sub-system's priority algorithm. This algorithm will be constantly informed not only of the state of the disinfecting robot (e.g., UV-C lamp operable or chemical supply level sufficient for different rooms or spaces) but also of the state of all automated shades in the multitude of rooms and spaces. In this way, the robot will not be dispatched to a room or space if the automated shade is not operational; in the case of a request to disinfect a room with an inoperable shade, the sub-system will post an error message and/or send an alert and send a command to the robot to proceed to the next room or space with an operable shade in the task queue.

In the case where the robot or robots are to autonomously disinfect a multitude of rooms, the communication mechanism needs to allow for these robots to selectively control the shades. This means that the robots need to be able to only lower, respectively raise, one specific shade at a time. To accomplish this, the communication protocol, in one embodiment, uses different control signals, e.g., signals that encode a digital identifier, signals that use different frequencies, or signals that use different temporal patterns. The controls device on each shade knows its own such signal pattern and will hence know when a received signal is intended for itself or a different shade. This is only necessary with certain communication protocols and when a multitude of shades are close enough to each other.

### Generalizations

In other possible embodiments of this invention, the shades are much larger than the width of a door frame and are mounted in such a way as to create sections inside a larger space, such that no significant amount of UV-C light can escape that section. Thereby the robot is able to section off an area, disinfect it, and then move on to the next area.

In another embodiment, the mechanism to close off the area of interest could be something other than a shade, e.g., an automated door, a garage door, or motorized room divider, that is similarly controlled by the robot.

In another possible embodiment, the robot itself carries the shades in a compacted format, deploys the shades at the room door before or after entering, depending on whether the room door opens inwards or outwards, and then detaches from it. On the way out, it reestablishes contact with these shades, puts them in their compact format again, and carries them away. One possible embodiment of this kind would be multiple panels that are connected with flexible joints, e.g., hinges, like folding screens. In this embodiment the robot could, e.g., use a fork-lift like mechanism to lift the panels up for transport, and lower them to the ground for deployment. The compact format in this case would be when the panels are folded, and in their deployed state they would be unfolded. The folding and unfolding can be realized via a servo-motor attached to one of two adjacent panels, while an arm that is rotated by the coil of the motor is attached to the other panel.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

### The Benefit

The System and Method inventions, herein described, enable robots to fully autonomously disinfect a large multitude of rooms and spaces without any human assistance can drastically reduce the rate of infections in hospitals and other in-door environments. Based on the presented invention, a fleet of robots can swarm these places and disinfect much more frequently than is currently possible when a human needs to be dispatched to each room to help the robot. As a result, pathogens, especially so-called superbugs, antibiotics-resistant bacteria like MERS and C. difficile, as well as pandemic-causing viruses like SARS-Cov-2, can be killed much more effectively, saving people from harm or death. Furthermore to a hospital operator, this solution can result in significant cost-reductions without sacrificing disinfection quality.

1. A method to disinfect indoor spaces comprising:
a mobile robot equipped with a disinfection mechanism; a motorized shade or multiple shades each comprising a motor, a control device and a communication device;
the shade or shades mounted such that running their motor enables changing from an “open” to a “closed” position and vice versa;
the shade or shades mounted such that when in the “open” position, the robot can enter the indoor space;
the shade or shades mounted such that when in the “closed” position, they prevent any significant amount of light or gas released inside the indoor space from leaving the indoor space;
each control device receiving “open” or “close” signals from the communication device and running the motor accordingly to transition the shade into the “open” or “closed” position;
each communication device receiving “open” or “closed” signals from the robot;
the robot comprising one or more sensors that collect sensor data;
the robot comprising one or more processors;
the robot comprising one or more communication devices;
a non-transitory computer-readable medium having instructions embodied thereon, the instructions, when executed by the one or more processors, perform operations comprising:
receiving a task to disinfect the indoor space;
navigating the robot into the indoor space to be disinfected;
detecting when the robot is inside the indoor space to be disinfected;
transmitting a “close” signal to the communication devices of the shade or shades;
detecting when the shade or shades are in the “closed” position;
activating the disinfection mechanism;
deactivating the disinfection mechanism;
transmitting an “open” signal to the communication devices of the shade or shades;
detecting when the shade or shades are in the “open” position;
navigating the robot out of the indoor space.

2. The method of claim 1, wherein the shades are mounted above all door-frames leading into the indoor space to be disinfected.

3. The method of claim 1, wherein the shades comprise rollup blinds.

4. The method of claim 1, wherein the shades comprise curtains.

5. The method of claim 1, wherein the shades are doors leading into the indoor space to be disinfected, equipped with the motor, control device and communication device such that they can be opened and closed via a signal received by the communication device.

6. The method of claim 1, wherein the shade material is impermeable to disinfecting chemicals and opaque to UV-C light while being transparent to light that is in the visible spectrum of humans.

7. The method of claim 1, wherein the communication devices of the robot and the shades communicate via the Internet.

8. The method of claim 1, wherein the communication devices of the robot and the shades communicate via Bluetooth.

9. The method of claim 1, wherein the communication devices of the robot and the shades communicate via a communication network that is internal to the building.

10. The method of claim 1, wherein the shade or shades have signage on them indicating that disinfection is in progress or are transparent to non-UV light such that a person can see that disinfection is in progress on the other side.

11. The method of claim 1, wherein each shade can be individually and unambiguously radio controlled utilizing a property such as a unique control address or radio transceiver frequency.

12. The method of claim 1, wherein the disinfection mechanism comprises an ultraviolet-C light source.

13. The method of claim 1, wherein the disinfection mechanism comprises a spraying mechanism capable of spraying germicidal chemicals.

14. A method to disinfect indoor spaces comprising:
a stationary robot equipped with a disinfection mechanism; a motorized shade or multiple shades each comprising a motor, a control device and a communication device;
the shade or shades mounted such that running their motor enables changing from an “open” to a “closed” position and vice versa;
the shade or shades mounted such that when in the “open” position, the robot can enter the indoor space;
the shade or shades mounted such that when in the “closed” position, they prevent any significant amount of light or gas released inside the indoor space from leaving the indoor space;
each control device receiving “open” or “close” signals from the communication device and running the motor accordingly to transition the shade into the “open” or “closed” position;
each communication device receiving “open” or “closed” signals from the robot;
the robot mounted inside the indoor space to be disinfected;
the robot comprising one or more sensors that collect sensor data;
the robot comprising one or more processors;
the robot comprising one or more communication devices;
a non-transitory computer-readable medium having instructions embodied thereon, the instructions, when executed by the one or more processors, perform operations comprising:
receiving a task to disinfect the indoor space;
transmitting a “close” signal to the communication devices of the shade or shades;
detecting when the shade or shades are in the “closed” position;
activating the robot's disinfection mechanism;
deactivating the robot's disinfection mechanism;
transmitting an “open” signal to the communication devices of the shade or shades;
detecting when the shade or shades are in the “open” position.

15. The method of claim 14, wherein the disinfection mechanism comprises an ultraviolet-C light source.

16. The method of claim 14, wherein the disinfection mechanism comprises a spraying mechanism capable of spraying germicidal chemicals.

17. A method to disinfect indoor spaces comprising:
a mobile robot equipped with a disinfection mechanism; a shade carried by the mobile robot;
a mounting mechanism for the shade such that when mounted, the shade prevents any significant amount of light or gas released inside the indoor space from leaving the indoor space;
the robot comprising one or more sensors that collect sensor data;
the robot comprising one or more processors;
the robot comprising one or more communication devices;
a non-transitory computer-readable medium having instructions embodied thereon, the instructions, when executed by the one or more processors, perform operations comprising:
receiving a task to disinfect the indoor space;
navigating the robot into the indoor space to be disinfected;
detecting when the robot is inside the indoor space to be disinfected;
the robot mounting the shade on the mounting mechanism;
activating the disinfection mechanism;
deactivating the disinfection mechanism;
the robot unmounting the shade from the mounting mechanism;
navigating the robot out of the indoor space.

18. The method of claim 17, wherein the mounting mechanism for the shade comprises a rail or hook.

19. The method of claim 17, wherein the disinfection mechanism comprises an ultraviolet-C light source.

20. The method of claim 17, wherein the disinfection mechanism comprises a spraying mechanism capable of spraying germicidal chemicals.
